(19) United States
(12) Patent Application Publication
(10) Pub. No.: US 2025/0327747 A1
(43) Pub. Date: Oct. 23, 2025

(54) SENSOR BOARD AND METHOD FOR DETERMINING A TYPE OF A BEVERAGE IN A CONTAINER AND A HEATING AND/OR FROTHING DEVICE WITH SUCH A SENSOR BOARD

(71) Applicant: LABRINTS BVBA, Puurs (BE)

(72) Inventor: Peter HERNOU, Puurs (BE)

(73) Assignee: LABRINTS BVBA, Puurs (BE)

(21) Appl. No.: 19/121,980

(22) PCT Filed: Oct. 20, 2023
(86) PCT No.: PCT/IB2023/060618

§ 371 Date: Apr. 17, 2025

(30) Foreign Application Priority Data
Oct. 21, 2022 (BE) BE2022/5848

(51) Int. Cl. G01N 21/3577; A47J 31/44; A47J 31/52; G01N 33/14

(52) U.S. Cl. CPC G01N 21/3577; A47J 31/4489; A47J 31/52; G01N 33/14

(57) ABSTRACT

A sensor board for determining a type of a beverage in a container, comprising a processing unit, at least one light source for emitting light, and at least one spectral sensor for collecting reflected light from the at least one light source, wherein the at least one light source has a spectrum with a peak between 780 nm and 950 nm and the at least one spectral sensor has a spectral response with a peak between 760 nm and 970 nm, wherein the said peak of the spectral response of the at least one spectral sensor deviates by no more than 20 nm from the peak of the at least one light source. A heating and/or frothing device for heating and/or frothing beverages. A method for determining a type of a beverage in a container.

### TECHNICAL FIELD

The invention relates to a sensor board for determining a type of a beverage in a container, more in particular a sensor board for a heating and/or frothing device for heating and/or frothing beverages. The invention also relates to a method for determining a type of a beverage in a container.

### PRIOR ART

When preparing beverages, more specifically when preparing beverages that are heated or frothed using a steam pipe, it is important to closely follow a specific recipe depending on the type of beverage being heated or frothed, such as milk or a plant-based alternative, during the heating and/or frothing. In a standard heating and/or frothing device, the type of beverage is visually determined by, for example, the bar staff who operate the device. With certain types of beverages this is not always only visually possible. In addition, bar staff could rely on the smell and taste of the beverage. For customers, it can be unpleasant and even unacceptable to see bar staff smelling or tasting the beverage while preparing the beverage. It is therefore necessary to keep the packaging close by in order to determine the type of beverage. When preparing multiple beverages quickly, whereby several containers with different but similar beverages may be prepared, a mistake can easily be made, resulting in the beverage being heated or frothed incorrectly and the beverage not being prepared optimally. In these cases, the heated or frothed beverage is often discarded and started again. This therefore results in product loss. It is therefore desirable to be able to determine a type of a beverage in a container in an automated manner.

EP 3 928 994 (EP '994) describes a method for printing on a beverage. The beverage is, for example, a cup of coffee or a glass of beer that is placed under a printer after preparation or tapping. A text and/or image is then printed on the surface of the printer. Depending on the type of beverage, a coffee-based or hop-based ink is used. EP '994 mentions the use of spectral information, more specifically the absorption of light with a wavelength of 430 nm, to determine the color of the beer, so that an adapted color of ink can be used.

EP '994 has the disadvantage that it is not suitable for distinguishing beverages that are usually heated and/or frothed with a steam pipe, such as milk, a plant-based alternative to milk, chocolate milk, etc. Similarly disadvantageous is that EP '994 is only intended for use with almost completely filled glasses or cups but is not suitable for use with a container that sometimes contains only a minimal amount of beverage for heating or frothing, which makes automatic recognition of the beverage more difficult. The method from EP '994 is therefore only suitable for use with beverages that have already been prepared.

US 2021/022547 (US '547) describes a monitoring system for a beverage making device. The monitoring system comprises a sensor board with a light source and a spectral sensor for collecting reflected light from the light source. US '547 has the disadvantage that it is mainly suitable for recognizing a type of coffee.

US 2005/022674 (US '674) relates to a semi-automatic device for preparing beverages. The device is suitable for recognizing packaging of ingredients for preparing beverages, but not for recognizing beverages themselves.

US 2015/0136991 (US '991) concerns a system for automatic detection in beverage vending machines. The detection is mainly aimed at determining the presence or absence or correct placement of a beverage supply container.

The present invention aims to solve at least some of the above problems or drawbacks.

### SUMMARY OF THE INVENTION

In a first aspect, the present invention concerns a sensor board for determining a type of a beverage in a container, such as milk or plant-based alternatives to milk, juices, espumas or soups, according to claim 1.

The sensor board is advantageous for an automated determination of the type of beverage in the container due to the presence of at least one light source with a spectrum that has a peak between 780 nm and 950 nm. By measuring and processing spectral values in the light from the at least one light source that is reflected by the beverage in the container to the spectral sensor, the type of beverage in the container can be determined. The applicants have surprisingly found that a light source with such a peak in the spectrum is very advantageous for distinguishing beverages used in preparing a beverage, and in particular advantageous for distinguishing, for example, milk and plant-based alternatives. The use of the spectral sensor with a spectral response with a peak that deviates at most 20 nm from the peak of the at least one light source is advantageous in determining spectral values from the reflected light because the spectral sensor has a high sensitivity in the vicinity of the peak of the at least one light source. Being able to automatically determine the type of the beverage in the container is very advantageous when preparing the beverage, and in particular when heating and/or frothing the beverage, in order to automatically adjust a recipe for preparing the drink in order to achieve optimal preparation. In addition, specific hygienic standards can be monitored, for example monitoring the freshness of a product. The sensor board is also beneficial for determining whether a beverage contains ingredients that appear on an allergen list or that could trigger intolerance reactions. Milk and plant-based alternatives to milk are not always easy to distinguish visually. Visually distinguishing between different types of milk or different plant-based alternatives is even more complex. Distinguishing whether a beverage has already undergone a previous treatment, such as pasteurization or skimming, is visually almost impossible. Thanks to the sensor board, bar staff do not have to smell or taste the beverage to determine the type of beverage. It is not necessary to keep the beverage packaging at hand to avoid mistakes. During busy periods, several containers with similar but different beverages can be set out to speed up the preparation of the beverages, without causing mistakes.

Preferred forms of the sensor board are shown in claims 2 to 8.

A specific preferred form concerns a sensor board according to claim 2.

This preferred form is advantageous for avoiding deviating results in determining the type of beverage in the container by reflections of the light emitted by the at least one source on side walls of the container. When preparing a beverage, the container is filled with only a very limited amount or on the contrary more beverage, depending on the beverage being prepared. This also means that there will be more or less reflections of the emitted light on the side walls of the container. These reflections influence the spectral values measured by the at least one spectral sensor. By measuring the level of the beverage in the container or by measuring the volume of the beverage in the container, from which the level can be calculated, the influence of the reflections on the side walls of the container can be taken into account, allowing for a robust determination of the type of beverage in the container.

In a second aspect, the present invention relates to a heating and/or frothing device according to claim 9. Due to the presence of a sensor board according to the first aspect, this device is very advantageous for automatically adjusting and setting a recipe for the preparation of the beverage based on the type of beverage in the container, which is automatically determined using the sensor board. This prevents errors due to an incorrect visual determination of the type of beverage by the bar staff when preparing the beverage. It is also not necessary to keep a beverage container at hand to determine the beverage in the container. In addition, the bar staff can work together when it is very busy. For example, a first member of the bar staff or a cobot can set out multiple containers with similar, but different beverages, while a second member of the bar staff effectively prepares the beverage by heating and/or frothing the beverage in the container. Because the type of beverage is automatically detected, the recipe is automatically adjusted so that the second member of the bar staff optimally heats and/or froths the beverage in the container without even knowing the type of beverage in the container in advance.

A preferred form concerns a heating and/or frothing device according to claim 10.

The actuator for changing a mutual position between the platform for supporting the container and the sensor board is advantageous for obtaining a first fixed predetermined distance between the at least one light source and an upper liquid surface in the container and a second fixed predetermined distance between the at least one spectral sensor and the aforementioned upper liquid surface. This is advantageous because as a result reflections on the side walls of the container of the light emitted by the at least one light source will always have an approximately similar influence on the spectral values measured by the spectral sensor, regardless of how much beverage is present in the container. It is also advantageous that approximately a similar amount of light emitted by the at least one light source will always reach the upper liquid surface, which means that there will be fewer variations due to ambient light in the spectral values obtained. This results in a robust determination of the type of beverage in the container.

In a third aspect, the present invention relates to a method according to claim 11.

This method is advantageous for an automated determination of the type of beverage in the container by using the at least one light source with a spectrum that has a peak between 780 nm and 950 nm. By determining and processing the spectral values in the light from the at least one light source that is reflected by the beverage in the container to the spectral sensor, the type of beverage in the container can be determined. A light source with a peak between 780 nm and 950 nm is very advantageous for distinguishing beverages used in preparing a beverage, and particularly advantageous for distinguishing, for example, milk and plant-based alternatives. The use of the spectral sensor with a spectral response with a peak that deviates at most 20 nm from the peak of the at least one light source is advantageous in determining spectral values from the reflected light because the spectral sensor has a high sensitivity in the vicinity of the peak of the at least one light source. Being able to automatically determine the type of the beverage in the container is very advantageous when preparing the beverage, and in particular when heating and/or frothing the beverage, in order to automatically adjust a recipe for preparing the drink in order to achieve optimal preparation, while avoiding errors.

Preferred forms of the method are described in dependent claims 12 to 15.

In a fourth aspect, the invention relates to a method for heating and/or frothing a beverage in a container, such as milk or plant-based alternatives to milk, juices, espumas or soups, comprising the steps of determining a type of the beverage in the container using a sensor board according to the first aspect and subsequently heating and/or frothing the beverage in the container according to a predetermined recipe.

This method is advantageous because a beverage is optimally heated and/or frothed according to a recipe in accordance with the type of beverage in the container, thereby avoiding errors.

### DETAILED DESCRIPTION

Unless otherwise defined, all terms used in the description of the invention, including technical and scientific terms, have the meaning as commonly understood by a person skilled in the art to which the invention pertains. For a better understanding of the description of the invention, the following terms are explained explicitly.

In this document, “a” and “the” refer to both the singular and the plural, unless the context presupposes otherwise. For example, “a segment” means one or more segments.

The terms “comprise”, “comprising”, “consist of”, “consisting of”, “provided with”, “include”, “including”, “contain”, “containing”, are synonyms and are inclusive or open terms that indicate the presence of what follows, and which do not exclude or prevent the presence of other components, characteristics, elements, members, steps, as known from or disclosed in the prior art.

Quoting numerical intervals by endpoints comprises all integers, fractions and/or real numbers between the endpoints, these endpoints included.

Furthermore, the terms “first”, “second”, “third” and the like are used in the description and in the claims to distinguish between like elements and not necessarily to describe a sequential or chronological order unless specified. It is to be understood that the terms so used are interchangeable under appropriate circumstances and that the embodiments of the invention described herein may operate in sequences other than those described or illustrated herein.

In the context of this document, “container” means a container for beverages. Non-limiting examples of suitable materials for the container are ceramic, porcelain, metal, glass, plastic, cardboard, etc. The container is preferably a metal container. A non-limiting example of a container is a jug, which is provided with a spout and preferably also with a handle. Another non-limiting example is a cup, preferably a cardboard or plastic cup that can be closed with a lid with a drink opening. In the context of this document, an LED is a Light Emitting Diode.

In the context of this document, spectral response of a spectral sensor means the response of the spectral sensor to light of equal power as a function of the wavelength of the light.

In the context of this document, determining a type of a beverage means that a beverage is identified and classified into a category based on the properties of the beverage. The type of the beverage then corresponds to the category in which the beverage was classified. Non-limiting examples of properties of beverages are fat content, color, composition, etc. Non-limiting examples of categories are milk and plant-based alternatives to milk. These categories can be defined even more specifically, such as skimmed milk, semi-skimmed milk, whole milk, almond milk, rice milk, soy milk, coconut milk, hazelnut milk, etc.

In a first aspect, the invention concerns a sensor board for determining a type of a beverage in a container, such as milk or plant-based alternatives to milk, juices, espumas or soups.

According to a preferred embodiment, the sensor board comprises a processing unit, at least one light source for emitting light and at least one spectral sensor for collecting reflected light from the at least one light source.

The sensor board comprises a rigid printed circuit board, a flexible printed circuit board or a combination of both. The sensor board is preferably a single printed circuit board. Alternatively, the sensor board is composed of multiple printed circuit boards, wherein the multiple printed circuit boards are connected to each other using connectors and/or cables.

The processing unit is a processor or a microcontroller. Preferably the processing unit is a microcontroller. Non-limiting examples of suitable microcontrollers are an ARM Cortex M4 or an ARM Cortex M7. The processing unit is preferably coupled communicatively to the at least one spectral sensor for setting the at least one spectral sensor and for reading spectral values determined by the at least one spectral sensor. If the sensor board comprises more than one spectral sensor, the processing unit is preferably communicatively coupled to each of the spectral sensors. The processing unit is directly or indirectly coupled communicatively to the at least one spectral sensor. Indirectly communicatively coupled means that the processing unit communicates with another chip, which in turn is directly or indirectly communicatively coupled to the at least one spectral sensor. Preferably, the processing unit is directly communicatively coupled to the at least one spectral sensor via a serial connection, such as UART or I² C.

The at least one light source is preferably an LED. If the sensor board comprises more than one light source, each light source is preferably an LED. Preferably, the LED has a power of at least 0.50 W, more preferably at least 0.75 W, even more preferably at least 1.00 W and even more preferably at least 1.25 W. Preferably, the LED has a power of at most 3.50 W, more preferably at most 3.00 W, even more preferably at most 2.50 W and even more preferably at most 2.00 W. Alternatively, the light source is a laser. The laser preferably has a power that corresponds to the previously mentioned powers for an LED. The at least one light source preferably comprises a lens for directing the emitted light to the beverage in the container. If the sensor board comprises more than one light source, each light source preferably comprises a lens for directing the emitted light towards the beverage in the container. The at least one light source has a spectrum with a peak between 780 nm and 950 nm. Specifically advantageous values are 830 nm and 940 nm. Preferably, the peak in the spectrum of the at least one light source between 780 nm and 950 nm is a global maximum in the spectrum. If the sensor board comprises more than one light source, then at least one light source has a spectrum with a peak between 780 nm and 950 nm. Multiple light sources in the spectrum may have a peak between 780 nm and 950 nm, where the peaks between 780 nm and 950 nm of the multiple light sources may or may not be the same.

The at least one spectral sensor is preferably a sensor chip. This is advantageous because of the limited space required by a sensor chip on the sensor board and the limited cost of the sensor chip. The at least one spectral sensor is suitable for capturing light that has been emitted by the at least one light source and reflected on an upper liquid surface formed by the beverage in the container. If the sensor board comprises multiple spectral sensors, the multiple spectral sensors are preferably part of a single chipset. Non-limiting examples of suitable spectral sensors are AS7265 and AS7341 from Osram. The at least one spectral sensor has a spectral response with a peak between 760 nm and 970 nm. The said peak between 760 nm and 970 nm of the spectral response of the at least one spectral sensor deviates by a maximum of 20 nm from the said peak between 780 nm and 950 nm in the spectrum of the at least one light source, preferably at most 18 nm, more preferably at most 16 nm, even more preferably at most 14 nm and even more preferably at most 12 nm. Preferably, the peak in the spectral response of the at least one spectral sensor between 760 nm and 970 nm is a global maximum in the spectral response. If the sensor board comprises more than one spectral sensor, then at least one spectral sensor has a spectral response with a peak between 760 nm and 970 nm. Multiple spectral sensors may have a peak in the spectral response between 760 nm and 970 nm, where the peaks between 760 nm and 970 nm of the multiple spectral sensors may or may not be the same.

The sensor board is advantageous for automated determination of the type of beverage in the container. A beverage itself has a spectral response. When light falls on the beverage, the beverage will let some of the light through, reflect some and absorb some. This depends on the wavelength of the light. “Spectral response” of the beverage refers to the part of the light that is reflected. The spectral response of the beverage also depends on the composition of the beverage. Based on the spectral response it is therefore possible to distinguish certain beverages from each other. Distinguishing beverages can be done in a single step or in several steps, whereby first a main category and then one or more subcategories for the beverages are determined. Measuring the spectral response of the beverage over a full range from, for example, 400 nm to 970 nm, i.e. the visible light and the near-infrared, is only feasible with expensive spectrometers and light sources with a very broad spectrum. This requires specific, expensive equipment and takes a lot of time. This is not feasible for automated determination of the type of a beverage in a container when preparing a beverage. The applicants have surprisingly found that a light source having a spectrum with a peak between 780 nm and 950 nm is very advantageous for distinguishing beverages used in the preparation of a beverage, and particularly advantageous for distinguishing, for example, milk and plant-based alternatives. This is particularly advantageous because by measuring the spectral response at a single point between 780 nm and 950 nm, a number of types of beverages can already be determined quickly and automatically with a simple sensor board. A light source with a spectrum with a peak between 780 nm and 950 nm is not only advantageous for distinguishing, for example, milk and plant-based alternatives, but also for at least partially distinguishing milk based on fat content. The use of the spectral sensor with a spectral response with a peak that deviates at most 20 nm from the peak of the at least one light source is advantageous in determining spectral values from the reflected light because the spectral sensor has a high sensitivity in the vicinity of the peak of the at least one light source. Being able to automatically determine the type of the beverage in the container is very advantageous when preparing the beverage, and in particular when heating and/or frothing the beverage, in order to automatically adjust a recipe for preparing the drink in order to achieve optimal preparation. In addition, specific hygienic standards can be monitored, for example monitoring the freshness of a product. The sensor board is also beneficial for determining whether a beverage contains ingredients that appear on an allergen list or that could trigger intolerance reactions. Milk and plant-based alternatives to milk are not always easy to distinguish visually. Visually distinguishing between different types of milk or different plant-based alternatives is even more complex. Determining a fat content of milk visually is not at all easy. Distinguishing whether a beverage has already undergone a previous treatment, such as pasteurization or skimming, is visually almost impossible. Thanks to the sensor board, bar staff do not have to smell or taste the beverage to determine the type of beverage. It is not necessary to keep the beverage packaging at hand to avoid mistakes. During busy periods, several containers with similar but different drinks can be set out to speed up the preparation of the beverages, without causing mistakes.

According to a preferred embodiment, the sensor board comprises a measuring sensor for measuring a level and/or a volume of the beverage in the container. The measuring sensor is, for example, a ToF (Time of Flight) sensor that measures a distance between an upper liquid surface formed by the beverage in the container and the ToF sensor. The measuring sensor is, for example, a sensor that is immersed in the beverage in the container. Preferably, the measuring sensor is a ToF sensor. This is advantageous because there is no contact between the measuring sensor and the beverage in the container. The processing unit is preferably communicatively coupled directly or indirectly to the measuring sensor. Preferably, the processing unit is directly communicatively coupled to the measuring sensor via a serial connection, such as UART or I² C.

Other non-limiting examples of suitable measuring sensors for measuring a level and/or a volume of the beverage in the container are a laser and a camera. A laser is suitable for measuring a distance between the level of the beverage in the container and the sensor board. A camera is suitable for estimating the level and/or volume of the beverage in a known container. With a known container, it can be estimated on the basis of a camera image how far the beverage comes from an upper edge of the known container or another reference point in the known container, from which the level and/or volume of the beverage in the known container can be determined.

This embodiment is advantageous for avoiding deviating results in determining the type of beverage in the container by reflections of the light emitted by the at least one light source on side walls of the container. When preparing a beverage, the container is filled with only a very limited amount or on the contrary much more of the beverage, depending on the beverage being prepared. This also means that there will be more or less reflections of the emitted light on the side walls of the container. These reflections influence the spectral values measured by the at least one spectral sensor. The reflections on the side walls of the container are not representative of the beverage in the container. By measuring the level of the beverage in the container or by measuring the volume of the beverage in the container, from which the level is calculated, the influence of the reflections on the side walls of the container can be taken into account, allowing for a robust determination of the type of beverage in the container.

According to a preferred embodiment, the sensor board comprises a temperature sensor for measuring the temperature of the beverage in the container. The temperature sensor is, for example, a temperature sensor that is immersed in the beverage in the container. The temperature sensor is, for example, a thermometer that measures the temperature of the container. The temperature of the container is a measure of the temperature of the beverage in the container. Preferably, the temperature sensor is an optical sensor, based on infrared measurements. This is advantageous because as a result there is no contact between the temperature sensor and the beverage in the container and because the temperature of the beverage is measured directly, instead of indirectly by measuring the temperature of the container. The processing unit is preferably communicatively coupled directly or indirectly to the temperature sensor. Preferably, the processing unit is directly communicatively coupled to the temperature sensor via a serial connection, such as UART or I² C.

This embodiment is advantageous for avoiding deviating results when determining the type of the beverage in the container by the temperature of the beverage in the container. Properties of the beverage in the container may depend on the temperature of the beverage. For example, a beverage may expand when heated, which reduces the specific gravity of the beverage. This can have an influence on the spectral response of the beverage in the container. By measuring the temperature of the beverage in the container, the influence of the temperature of the beverage on its spectral response can be taken into account, allowing for a robust determination of the type of beverage in the container.

This embodiment is additionally advantageous when using the sensor board in a heating and/or frothing device according to the second aspect. Depending on the type of beverage, a beverage cannot be heated to the same degree without the beverage breaking down, causing loss of flavor or quality, or froth disintegration during frothing. In this case, the temperature sensor is not only advantageous for correctly determining the beverage, but at the same time allows to determine whether the beverage has a suitable temperature.

According to a preferred embodiment, the sensor board comprises at least two light sources. A first light source has a spectrum with a peak between 780 nm and 849 nm. A specifically advantageous value is 830 nm. Preferably, the peak in the spectrum of the first light source between 780 nm and 849 nm is a global maximum in the spectrum. A specifically advantageous value is 830 nm. A second light source has a spectrum with a peak between 850 nm and 950 nm. A specifically advantageous value is 940 nm. Preferably, the peak in the spectrum of the first light source between 850 nm and 950 nm is a global maximum in the spectrum. One or more spectral sensors have a spectral response with a first peak. The first peak deviates by no more than 20 nm from the said peak between 780 nm and 849 nm of the spectrum of the first light source. One or more spectral sensors have a spectral response with a second peak. The second peak deviates by no more than 20 nm from the said peak between 850 nm and 950 nm of the spectrum of the second light source. In the event the sensor board comprises only one spectral sensor, it is clear that the single spectral sensor has a spectral response with the first peak and the second peak. In case the sensor board comprises multiple spectral sensors, a first spectral sensor may have a spectral response with the first peak and a second spectral sensor may have a spectral response with the second peak or a single spectral sensor may have a spectral response with the first peak and the second peak. Similarly, it is possible for multiple spectral sensors to have a spectral response with the first peak, the second peak or the first peak and the second peak.

This embodiment is advantageous for a more accurate determination of the type of the beverage in the container. The applicants surprisingly found that with this embodiment a better distinguishing capacity is achieved between different types of milk with different compositions, such as fat content, between milk and plant-based alternatives and between plant-based alternatives among themselves. A deviation between the first peak and the said peak between 780 nm and 849 nm of the spectrum of the first light source and between the second peak and the said peak between 850 nm and 950 nm of the spectrum of the second light source of not more than 20 nm is advantageous in determining spectral values from the reflected light because one or more spectral sensors in the vicinity of the first peak and the second peak have a high sensitivity.

According to a preferred embodiment, the at least one light source is current-controlled. If the sensor board comprises multiple light sources, all light sources are preferably current-controlled. A current intensity for the at least one light source can be adjusted from the processing unit. For example, the current intensity is adjustable because the processing unit is directly or indirectly communicatively coupled to a driver for the at least one light source. If the sensor board comprises multiple light sources, the processing unit is preferably directly or indirectly communicatively coupled to each driver of each light source.

Preferably the current is at least 200 mA, more preferably at least 250 mA, even more preferably at least 300 mA and even more preferably at least 350 mA.

Preferably, the current is at most 850 mA, more preferably at most 800 mA, even more preferably at most 750 mA and even more preferably at most 700 mA.

This embodiment is particularly advantageous for calibration of the sensor board. By calibrating the sensor board, it can be obtained that similar spectral values are obtained by the spectral sensors for similar beverages, allowing a robust determination of the type of beverage in the container. Spectral sensors usually have an adjustable gain for their spectral response. This adjustable gain is usually the same for each peak of the spectral response. By adjusting the current intensity for each light source separately, the spectral response for each peak in the spectral response can be indirectly influenced. This embodiment is particularly advantageous if the sensor board comprises multiple light sources. This embodiment is also advantageous for obtaining similar spectral values during the life of the sensor board. A light source emits less light as it ages. By increasing the current intensity based on the number of active hours of the light source, i.e. the number of hours during which the light source has emitted light, it is possible to obtain a constant light output for the light source during the life of the sensor board. This embodiment is also particularly advantageous for automatic strengthening or weakening of a light source. If the current intensity is set too low, depending on the type of beverage in the container, minimal spectral values may be obtained at certain wavelengths. If the current intensity is set too high, depending on the type of beverage in the container, maximal spectral values may be obtained at certain wavelengths. In both cases, information may be lost and the spectral values may not have sufficient distinguishing capacity to successfully determine the type of beverage in the container. By automatically amplifying or weakening the current strength, spectral values can be obtained that are not minimal or maximal and which still provide sufficient distinguishing capacity.

According to a preferred embodiment, the sensor board comprises a first additional light source. The first additional light source has a spectrum with a peak between 410 nm and 480 nm. A specifically advantageous value is 420 nm. Preferably, the peak in the spectrum of the first additional light source between 410 nm and 480 nm is a global maximum in the spectrum. The spectral response of one or more spectral sensors has a peak between 400 nm and 500 nm. The mentioned peak between 400 nm and 500 nm deviates at most 20 nm from the peak of the first additional light source.

This embodiment is advantageous for a more accurate determination of the type of the beverage in the container. The applicants surprisingly found that with this embodiment a better distinguishing capacity is achieved between different types of milk with a similar composition, such as a similar fat content, but from a different manufacturer, between plant-based alternatives among themselves, such as coconut milk, soy milk, hazelnut milk, etc., and between similar plant-based alternatives from different manufacturers. This embodiment is also advantageous for distinguishing beverages with a small color difference. A deviation of at most 20 nm between the peak of the first additional light source and the said peak between 400 nm and 500 nm is advantageous when determining spectral values from the reflected light because one or more spectral sensors placed in the vicinity of the said peak of the first additional light source have a high sensitivity.

According to a preferred embodiment, the sensor board comprises a second additional light source. The second additional light source has a spectrum with a peak between 720 nm and 760 nm. A specifically advantageous value is 760 nm. Preferably, the peak in the spectrum of the second additional light source between 720 nm and 760 nm is a global maximum in the spectrum. The spectral response of one or more spectral sensors has a peak between 700 nm and 800 nm. The mentioned peak between 700 nm and 800 nm deviates at most 20 nm from the peak of the second additional light source.

This embodiment is advantageous for a more accurate determination of the type of the beverage in the container. The applicants surprisingly found that this embodiment achieves a better distinguishing capacity between a fat content of different beverages, but also a better distinguishing capacity between milk and plant-based alternatives and between plant-based alternatives among themselves. This embodiment is particularly advantageous in combination with a previously described embodiment in which the sensor board comprises a first light source and a second light source. A deviation of at most 20 nm between the peak of the second additional light source and the said peak between 700 nm and 800 nm is advantageous when determining spectral values from the reflected light because one or more spectral sensors placed in the vicinity of the said peak of the second additional light source have a high sensitivity.

According to a preferred embodiment, the sensor board comprises a third additional light source. The third additional light source emits white light. The third additional light source is preferably a white LED. The third additional light source preferably has a spectrum of 500 nm to 700 nm, whereby at a wavelength of 500 nm and at a wavelength of 700 nm light is emitted that is at least 5% of a maximum intensity of the light between 500 nm and 700 nm. A joint spectral response of all spectral sensors has at least eight peaks between 400 nm and 700 nm. The joint spectral response is a summation of the spectral response of each individual spectral sensor. If the sensor board comprises only one spectral sensor, the joint spectral response is the spectral response of the at least one spectral sensor. Between 400 nm and 700 nm there is a spectral distance between two adjacent peaks of the joint response of at most 50 nm, preferably at most 45 nm, even more preferably at most 40 nm. This embodiment is advantageous for improving an overall distinguishing capacity based on the spectral values using white light. This embodiment is particularly advantageous if there is not sufficient distinguishing capacity between two beverages based on any of the previously described embodiments, as a result of which the type of the beverage in the container cannot be determined with enough certainty. A joint spectral response with at least eight peaks between 400 and 700 nm with a spectral distance of at most 50 nm is sufficient to adequately measure a spectral response of a beverage to determine the type of beverage in the container.

In a second aspect, the invention concerns a heating and/or frothing device for heating and/or frothing beverages.

According to an embodiment, the heating and/or frothing device comprises a frame, a platform for supporting a container and a sensor board according to the first aspect for determining a type of beverage in the container. The heating and/or frothing device further comprises a heating means for heating the beverage in the container and/or a frothing means for frothing the beverage in the container. A non-limiting example of a heating means is an electric heating element. A non-limiting example of a frothing means is a rotary beater, wherein the rotary beater is preferably magnetically drivable. Alternatively, the rotary beater can be driven mechanically using a shaft. Preferably the heating means and the frothing means are combined, such as, for example, a steam pipe.

The at least one light source and the at least one spectral sensor of the sensor board face the platform. Preferably, an optical axis of the at least one light source and an optical axis of the at least one spectral sensor are placed transversely to the platform. As a result, the optical axis of the at least one light source and an optical axis of the at least one spectral sensor will automatically be placed transversely to an upper liquid surface formed by the beverage in the container.

This embodiment is very advantageous for automatically adjusting and setting a recipe for the preparation of the beverage based on the type of beverage in the container.

According to a further embodiment, the heating and/or frothing device comprises a display and a rotary beater. The rotary beater is interchangeable. The display is directly or indirectly communicatively coupled to the processing unit of the sensor board. For example, the display is indirectly coupled to the processing unit of the sensor board via a main processing unit of the heating and/or frothing device. The display is configured to display a type of rotary beater to be used in frothing the beverage.

This embodiment is advantageous for optimal frothing of the beverage. Depending on the type of beverage, for example whole milk or semi-skimmed milk, frothing the beverage with a certain type of rotary beater may or may not be successful. Using the sensor board, the type of beverage in the container can be automatically determined, after which a designated type of rotary beater is automatically displayed on the display with which the beverage is optimally frothed.

According to a preferred embodiment, the heating and/or frothing device comprises a frame, a platform for supporting a container and a steam pipe. The frame is a supporting structure for the heating and/or frothing device. Preferably, the heating and/or frothing device comprises a housing that is mounted around the frame. The platform is attached to the frame. The steam pipe is attached to the frame. The steam pipe comprises a nozzle for injecting water vapor or compressed gas into a beverage in the container for heating and/or frothing the beverage. The heating and/or frothing device comprises a sensor board according to the first aspect for determining a type of the beverage in the container.

The at least one light source and the at least one spectral sensor of the sensor board face the platform. Preferably, an optical axis of the at least one light source and an optical axis of the at least one spectral sensor are placed transversely to the platform. As a result, the optical axis of the at least one light source and an optical axis of the at least one spectral sensor will automatically be placed transversely to an upper liquid surface formed by the beverage in the container.

This embodiment is very advantageous for automatically adjusting and setting a recipe for the preparation of the beverage based on the type of beverage in the container. The type of beverage in the container is determined automatically using the sensor board. This prevents errors due to an incorrect visual determination of the type of beverage by the bar staff when preparing the beverage. It is also not necessary to keep a beverage container at hand to determine the beverage in the container. In addition, the bar staff can work together when it is very busy. For example, a first member of the bar staff or a cobot can set out multiple containers with similar, but different beverages, while a second member of the bar staff effectively prepares the beverage by heating and/or frothing the beverage in the container. Because the type of beverage is automatically detected, the recipe is automatically adjusted so that the second member of the bar staff optimally heats and/or froths the beverage in the container without even knowing the type of beverage in the container in advance. The heating and/or frothing device can heat and/or froth the beverage in the container fully automatically based on the recipe, allowing the second member of the bar staff to even perform another task during the heating and/or frothing.

According to a preferred embodiment, the heating and/or frothing device comprises an actuator for changing a mutual position between the platform and the sensor board. The actuator is communicatively coupled to the processing unit of the sensor board. The actuator is directly or indirectly communicatively coupled to the processing unit of the sensor board. For example, the actuator is indirectly coupled to the processing unit of the sensor board via a main processing unit of the heating and/or frothing device.

The actuator is advantageous to obtain a first fixed predetermined distance between the at least one light source and an upper liquid surface formed by the beverage in the container and a second fixed predetermined distance between the at least one spectral sensor and the said upper liquid surface. This is advantageous because as a result reflections on the side walls of the container of the light emitted by the at least one light source will always have an approximately similar influence on the spectral values measured by the spectral sensor, regardless of how much beverage is present in the container. It is also advantageous that approximately a similar amount of light emitted by the at least one light source will always reach the upper liquid surface, resulting in fewer variations due to ambient light. This results in a robust determination of the type of beverage in the container. It is clear that if the sensor board comprises multiple light sources and/or multiple spectral sensors, these multiple light sources and/or multiple spectral sensors preferably have a fixed position relative to each other, so that the described advantage is also retained in these cases.

According to an embodiment, the heating and/or frothing device comprises a network connection. The network connection can be either a wired or a wireless network connection, such as an Ethernet connection or a WiFi connection. A network connection is advantageous for remotely updating software running on the sensor board's processing unit. This is particularly advantageous if new types of beverages are to be recognized by the sensor board, for example a new plant-based alternative to milk or a new manufacturer for a type of beverage. The network connection is also advantageous for automatically receiving orders, for example from a cash register system, so that a correct recipe for a beverage can be automatically selected and it can be verified using the sensor board whether the correct beverage will be heated and/or frothed. The network connection is also advantageous for transmitting spectral values of a beverage to a server, for example in the event that a beverage is not automatically recognized. The transmitted spectral values can then be used as training examples for a classification algorithm, as described further.

According to an embodiment, the heating and/or frothing device comprises an input means for entering data. The input means is, for example, a touch display, a keyboard or another suitable means. The input means is advantageous for entering a type of a beverage if the type of the beverage cannot be determined automatically with the aid of the sensor board. This embodiment is particularly advantageous in combination with a network connection as in a previously described embodiment. This allows the entered type of beverage to be sent to a server together with spectral values of the beverage, so that the spectral values can be used as annotated training examples for a classification algorithm. This embodiment is also advantageous for using the spectral values of the beverage together with the entered type of the beverage for training the classification algorithm on a processing unit of the heating and/or frothing device, as described in the next aspect. The input means is also particularly advantageous for entering a recipe for heating and/or frothing a beverage. This is particularly advantageous if the type of beverage cannot be determined automatically using the sensor board. This usually means that there is no recipe available for heating and/or frothing this type of beverage. By entering the recipe via the input means, beverages of an unknown type can also be heated and/or frothed with the heating and/or frothing device and the entered recipe can be associated with the entered type, so that afterwards this type of beverage can be automatically heated and/or frothed.

In a third aspect, the invention concerns a method for determining a type of a beverage in a container, such as milk or plant-based alternatives to milk, juices, espumas or soups.

According to a preferred embodiment, the method comprises the steps of:
illuminating the beverage in the container with light from at least one light source,
capturing light reflected by the beverage from the at least one light source using at least one spectral sensor,
processing spectral values of the reflected light determined by the at least one spectral sensor using a processing unit for determining the type of the beverage in the container.

The at least one light source has a spectrum with a peak between 780 nm and 950 nm. The at least one spectral sensor has a spectral response with a peak between 760 nm and 970 nm. The said peak of the spectral response of the at least one spectral sensor deviates at most 20 nm from the peak of the at least one light source. The at least one light source and the at least one spectral sensor are directed towards the beverage in the container. Preferably, an optical axis of the at least one light source and an optical axis of the at least one spectral sensor are placed transversely to an upper liquid surface formed by the beverage in the container. Preferably, the at least one light source illuminates the beverage for at least 50 ms, more preferably at least 75 ms, even more preferably at least 90 ms. This is advantageous because it means that the beverage is illuminated for a sufficiently long time so that the at least one spectral sensor can determine a stable spectral value. Preferably, the at least one light source illuminates the beverage for a maximum of 2.0 s, more preferably a maximum of 1.5 s and even more preferably a maximum of 1.2 s. This is advantageous to automatically determine the type of beverage in the container as quickly as possible. If more than one light source is used when carrying out the method, the different light sources can illuminate the beverage in the container both sequentially and simultaneously. Preferably, there is at least 100 ml of beverage in the container. This is advantageous to avoid light reflecting off the bottom of the container and interfering with the determination of the spectral response of the beverage.

Determining the type of the beverage can be done in a single step or in several steps, whereby first a main category and then one or more subcategories for the beverage are determined.

This method is advantageous for an automated determination of the type of beverage in the container by using the at least one light source with a spectrum that has a peak between 780 nm and 950 nm. By determining and processing the spectral values in the light from the at least one light source that is reflected by the beverage in the container to the spectral sensor, the type of beverage in the container can be determined. A light source with a peak between 780 nm and 950 nm is very advantageous for distinguishing beverages used in preparing a beverage, and particularly advantageous for distinguishing, for example, milk and plant-based alternatives. The use of the spectral sensor with a spectral response with a peak that deviates at most 20 nm from the peak of the at least one light source is advantageous in determining spectral values from the reflected light because the at least one spectral sensor has a high sensitivity in the vicinity of the peak of the at least one light source. Being able to automatically determine the type of the beverage in the container is very advantageous when preparing the beverage, and in particular when heating and/or frothing the beverage, in order to automatically adjust a recipe for preparing the drink in order to achieve optimal preparation, while avoiding errors.

According to a preferred embodiment, before illuminating the beverage in the container with light from the at least one light source, the method comprises a first additional step of measuring a level and/or a volume of the beverage in the container using a measuring sensor. The beverage in the container forms an upper liquid surface. The first additional step is followed by a second additional step. In the second additional step, the at least one light source and the upper liquid surface are positioned at a predetermined first distance relative to each other. In the second additional step, the at least one spectral sensor and the upper liquid surface are positioned at a predetermined second distance relative to each other. The first distance and the second distance are measured in a direction transverse to the upper liquid surface.

It is clear that if the at least one light source and the at least one spectral sensor have a fixed position relative to each other, by positioning the at least one light source and the upper liquid surface relative to each other at a predetermined first distance, the at least one spectral sensor and the upper liquid surface are also positioned at a predetermined second distance relative to each other. This embodiment also applies mutatis mutandis if multiple light sources and multiple spectral sensors are used when carrying out the method.

This embodiment is advantageous because as a result reflections on the side walls of the container of the light emitted by the at least one light source will always have an approximately similar influence on the spectral values measured by the spectral sensor, regardless of how much beverage is present in the container. It is also advantageous that approximately a similar amount of light emitted by the at least one light source will always reach the upper liquid surface, resulting in fewer variations due to ambient light. This results in a robust determination of the type of beverage in the container.

For example, this embodiment of the method is carried out using a heating and/or frothing device according to the second aspect, wherein the heating and/or frothing device comprises an actuator for changing the mutual position between the platform and the sensor board, as in a previously described embodiment of the heating and/or frothing device. In the second additional step, the platform is moved to the sensor board until the at least one light source and the upper liquid surface are positioned at the first predetermined distance, and the at least one spectral sensor and the upper liquid surface are positioned at the second predetermined distance from each other. For example, the platform is moved towards the sensor board until the nozzle of the steam pipe touches the upper liquid surface.

Preferably, the second additional step is only carried out after the level and/or volume of the beverage in the container, measured using the measuring sensor, is higher than a first predetermined value and lower than a second predetermined value. This is advantageous because there should not be too much or too little beverage in the container for heating and/or frothing a beverage. This is additionally advantageous because if the amount of beverage in the container is too small, an incorrect type of beverage may be determined in the container, for example because a bottom of the container becomes too visible and disrupts the expected reflections of the light.

According to a preferred embodiment, before processing the spectral values of the reflected light determined by the at least one spectral sensor with the aid of the processing unit, the method comprises a third additional step, in which the temperature of the beverage in the container is measured with the aid of a temperature sensor. This embodiment can, but does not have to, be advantageously combined with the previously described embodiment.

This embodiment is advantageous for avoiding deviating results when determining the type of the beverage in the container by the temperature of the beverage in the container. Properties of the beverage in the container may depend on the temperature of the beverage. For example, a beverage may expand when heated, which reduces the specific gravity of the beverage. This can have an influence on the spectral response of the beverage in the container. By measuring the temperature of the beverage in the container, the influence of the temperature of the beverage on its spectral response can be taken into account, allowing for a robust determination of the type of beverage in the container.

According to a preferred embodiment, when processing the spectral values of the reflected light determined by the at least one spectral sensor, a classification algorithm is executed on the processing unit. If multiple spectral sensors are used when carrying out the method, all spectral values determined by all spectral sensors are preferably processed by the classification algorithm on the processing unit.

The classification algorithm was trained using a multitude of training examples of beverages. Non-limiting examples of training examples are skimmed milk, semi-skimmed milk, whole milk, soy milk, coconut milk, hazelnut milk, etc. For each of the training examples, spectral values are obtained by illuminating each of the training examples with light from the at least one light source and collecting light reflected from each of the training examples using the at least one spectral sensor. If multiple spectral sensors and/or multiple light sources are used in the implementation of the method, spectral values for the training examples are preferably obtained with all spectral sensors and/or for all light sources. Non-limiting examples of suitable classification algorithms are Support Vector Machine (SVM) and Random Forest. Preferably, the classification algorithm is an SVM algorithm. Preferably, the classification algorithm is trained on a server or computer. Alternatively, the classification algorithm is trained on the processing unit, wherein the processing unit is included in a heating and/or frothing device according to the second aspect.

This embodiment is advantageous because, by using the training examples, a classification algorithm is obtained that robustly determines the type of the beverage in the container and that can easily be expanded for new types of beverages by adding training examples.

According to a further embodiment, each of the training examples is in the same container during the acquisition of the spectral values as during the determination of the type of the beverage.

This embodiment is particularly advantageous because the spectral values for the training examples can be determined in the same container that is used during the determination of the type of beverage when performing the method. This automatically takes into account the shape and material of the container, and therefore reflections on the side walls of the container. This embodiment is particularly advantageous in combination with a previously described embodiment, in which the at least one light source is positioned at a first predetermined distance and the at least one spectral sensor at a second predetermined distance relative to the upper liquid surface.

According to an embodiment, the plurality of training examples comprises a set of identical beverages at a different temperature. This embodiment is advantageous because it automatically takes into account a possible different spectral response of a beverage at different temperatures. This embodiment is particularly advantageous in combination with a previously described embodiment wherein the temperature of the beverage in the container is measured for processing the spectral values.

According to an embodiment, spectral values are obtained for the plurality of training examples at different light intensities of the at least one light source. When using an LED, this is for example at different current intensities to control the LED. This is advantageous to automatically take into account possible differences in spectral response of beverages at different light intensities. This is particularly advantageous if, for example, an automatic strengthening or weakening of a light source is applied, as previously described for the sensor board.

According to an embodiment, the at least one light source and the at least one spectral sensor are calibrated. The at least one light source and the at least one spectral sensor are calibrated using an empty beverage container. This embodiment of the method can be carried out both with the aid of a heating and/or frothing device according to the second aspect and with the aid of a sensor board according to the first aspect. When carrying out the method using a heating and/or frothing device according to the second aspect, the empty container for beverages is the container that is used with the heating and/or frothing device. The empty container is preferably placed in such a way that the empty container shields the at least one light source and the at least one spectral sensor from external light. For example, when using a heating and/or frothing device according to the second aspect, wherein the heating and/or frothing device comprises an actuator for changing the mutual position between the platform and the sensor board, as in a previously described embodiment of the heating and/or frothing device, the platform will be moved towards the sensor board until the empty container is positioned against or very close to the sensor board.

This embodiment is advantageous for accommodating differences caused by production margins between light sources and differences due to production margins between spectral sensors. In the event of deviating measurement results from expected measurement results, for example, different current intensities can be set for the light sources, as previously described when calibrating the sensor board according to the first aspect. Alternatively, scaling factors for the measurement results can be determined in the event of deviating measurement results. This makes it possible to use the same training examples for all sensor boards. The calibration is preferably carried out before commissioning the sensor board according to the first aspect or the heating and/or frothing device according to the second aspect. The calibration can be repeated during use of the sensor board according to the first aspect or the heating and/or frothing device according to the second aspect, for example to compensate for aging effects on the at least one light source and/or the at least one spectral sensor.

In a fourth aspect, the invention concerns a method for heating and/or frothing a beverage in a container, such as milk or plant-based alternatives to milk, juices, espumas or soups.

According to a preferred embodiment, the method comprises the steps of determining a type of beverage in the container and subsequently heating and/or frothing the beverage in the container according to a predetermined recipe.

Determining the type of beverage in the container is carried out using a sensor board according to the first aspect. Determining the type of beverage in the container is preferably done by carrying out a method according to the third aspect.

Heating and/or frothing of the beverage preferably starts automatically after the type of beverage in the container has been determined. The predetermined recipe is automatically selected from a collection of recipes in accordance with the type of beverage in the container. This means, for example, that whole milk is determined as the type of beverage in the container and the beverage is to be frothed, the recipe for frothing whole milk is selected from the collection of recipes. Heating and/or frothing of the beverage is preferably done with the aid of a heating and/or frothing device according to the second aspect.

This method is advantageous because a beverage is optimally heated and/or frothed according to a recipe in accordance with the type of beverage in the container, thereby avoiding errors.

One skilled in the art will appreciate that a method according to the third aspect is preferably carried out with a sensor board according to the first aspect or a heating and/or frothing device according to the second aspect and that a sensor board according to the first aspect or a heating and/or frothing device according to the second aspect is preferably configured for carrying out a method according to the third aspect. Each feature described in this document, both above and below, can therefore relate to any of the four aspects of the present invention.

1-15. (canceled)

16. Sensor board for determining a type of a beverage in a container, such as milk or plant-based alternatives to milk, juices, espumas or soups, comprising a processing unit, at least one light source for emitting light, and at least one spectral sensor for collecting reflected light from the at least one light source, characterized in that the at least one light source has a spectrum with a peak between 780 nm and 950 nm and in that the at least one spectral sensor has a spectral response with a peak between 760 nm and 970 nm, wherein the said peak of the spectral response of the at least one spectral sensor deviates by no more than 20 nm from the peak of the at least one light source.

17. The sensor board according to claim 16, characterized in that the sensor board comprises a measuring sensor for measuring a level and/or a volume of the beverage in the container.

18. The sensor board according to claim 16, characterized in that the sensor board comprises a temperature sensor for measuring the temperature of the beverage in the container.

19. The sensor board according to claim 16, characterized in that the sensor board comprises at least two light sources, wherein a first light source has a spectrum with a peak between 780 nm and 849 nm and wherein a second light source has a spectrum with a peak between 850 nm and 950 nm, in that one or more spectral sensors have a spectral response with a first peak, wherein the first peak deviates at most 20 nm from the peak of the first light source and in that one or more spectral sensors have a spectral response with a second peak, wherein the second peak deviates at most 20 nm from the peak of the second light source.

20. The sensor board according to claim 16, characterized in that the at least one light source is current-controlled, wherein a current intensity for the at least one light source can be adjusted from the processing unit.

21. The sensor board according to claim 16, characterized in that the sensor board comprises a first additional light source, wherein the first additional light source has a spectrum with a peak between 410 nm and 480 nm, and in that the spectral response of one or more spectral sensors has a peak between 400 nm and 500 nm, wherein the said peak between 400 nm and 500 nm deviates at most 20 nm from the peak of the first additional light source.

22. The sensor board according to claim 16, characterized in that the sensor board comprises a second additional light source, wherein the second additional light source has a spectrum with a peak between 720 nm and 780 nm, and in that the spectral response of one or more spectral sensors have a peak between 700 nm and 800 nm, wherein the said peak between 700 nm and 800 nm deviates at most 20 nm from the peak of the second additional light source.

23. The sensor board according to claim 16, characterized in that the sensor board comprises a third additional light source, wherein the third additional light source emits white light and wherein a joint spectral response of all spectral sensors has at least twelve peaks between 400 nm and 700 nm, where between 400 nm and 700 nm there is a spectral distance of at most 50 nm between two adjacent peaks of the joint spectral response.

24. Heating and/or frothing device for heating and/or frothing beverages, comprising a frame, a platform attached to the frame for supporting a container, and a steam pipe attached to the frame, the steam pipe comprising a nozzle for injecting water vapor or compressed gas into a beverage in the container for heating and/or frothing of the beverage, characterized in that the heating and/or frothing device comprises a sensor board according to claim 16, for determining a type of beverage in the container.

25. The heating and/or frothing device according to claim 24, characterized in that the heating and/or frothing device comprises an actuator for changing a mutual position between the platform and the sensor board, wherein the actuator is communicatively coupled to the processing unit of the sensor board is.

26. Method for determining a type of a beverage in a container, such as milk or plant-based alternatives to milk, juices, espumas or soups, comprising:
illuminating the beverage in the container with light from at least one light source;
capturing light reflected by the beverage from the at least one light source using at least one spectral sensor;
processing spectral values of the reflected light determined by the at least one spectral sensor using a processing unit for determining the type of the beverage in the container;
characterized in that the at least one light source has a spectrum with a peak between 780 nm and 950 nm and in that the at least one spectral sensor has a spectral response with a peak between 760 nm and 970 nm, wherein the said peak of the spectral response of the at least one spectral sensor deviates by no more than 20 nm from the peak of the at least one light source.

27. The method according to claim 26, characterized in that the method, before illuminating the beverage in the container with light from the at least one light source, comprises a first additional step of measuring a level and/or a volume of the beverage in the container using a measuring sensor, wherein the beverage in the container forms an upper liquid surface, wherein the first additional step is followed by a second additional step, wherein in the second additional step the at least one light source and the upper liquid surface are positioned at a predetermined first distance from each other and wherein in the second additional step the at least one spectral sensor and the upper liquid surface are positioned at a predetermined second distance from each other.

28. The method according to claim 26, characterized in that the method comprises, before processing the spectral values of the reflected light determined by the at least one spectral sensor with the aid of the processing unit, a third additional step, in which the temperature of the beverage in the container is measured with the aid of a temperature sensor.

29. The method according to claim 26, characterized in that when processing the spectral values of the reflected light determined by the at least one spectral sensor, a classification algorithm is executed on the processing unit, wherein the classification algorithm is trained using a plurality of training examples of beverages, wherein spectral values are obtained for each of the training examples by illuminating each of the training examples with light from the at least one light source and collecting light reflected by each of the training examples using the at least one spectral sensor, wherein each of the training examples is in the same container during the obtaining of the spectral values as during the determination of the type of the beverage.

30. The method according to claim 29, characterized in that the plurality of training examples comprises a set of identical beverages at a different temperature.
